(19) United States
(12) Patent Application Publication
(10) Pub. No.: US 2026/0301886 A1
(43) Pub. Date: Oct. 1, 2026

(54) MACHINE LEARNING POTENTIAL FOR FUNCTIONAL ORGANIC MOLECULAR CRYSTALS

(71) Applicant: University of Kentucky Research Foundation, Lexington, KY (US)

(72) Inventors: Chad Risko, Lexington, KY (US); Vinayak Bhat, Columbia, SC (US)

(21) Appl. No.: 19/694,408

(22) Filed: Jun. 1, 2026

Related U.S. Application Data
(63) Continuation-in-part of application No. 19/193,548, filed on Apr. 29, 2025.
(60) Provisional application No. 63/640,174, filed on Apr. 29, 2024.
(60) Provisional application No. 63/814,782, filed on May 30, 2025.

(51) Int. Cl. G16C 20/70; G06N 3/042; G16C 20/20; G16C 20/30; G16C 20/80

(52) U.S. Cl. CPC G16C 20/70; G06N 3/042; G16C 20/20; G16C 20/30; G16C 20/80

(57) ABSTRACT

This disclosure is directed to machine learning-based methods for modelling energies, forces, and stress tensors of organic molecular crystals. Methods for training a machine learning (ML) potential for analysis of crystal surface energies and crystal morphologies, and workflows using such ML potentials for predicting a surface energy and a crystal morphology and for modelling crystal structure relaxation of an organic molecular crystal are disclosed.

### RELATED APPLICATIONS

This application claims priority to U.S. provisional patent application S.N. 63/814,782 filed on May 30, 2025, and is a continuation-in-part patent application of U.S. patent application Ser. No. 19/193,548 filed on Apr. 29, 2025 which claims priority to U.S. provisional patent application Ser. No. 63/640,174 filed on Apr. 29, 2024, the entirety of the disclosures of each of which are incorporated herein in their entirety by reference.

### GOVERNMENT INTEREST

This invention was made with government support under award numbers DMR-1627428 and 2323422 awarded by the National Science Foundation Designing Materials to Revolutionize and Engineer our Future (NSF DMREF) and the National Institute of General Medical Sciences of the National Institutes of Health, Award No. P20GM103499. The government has certain rights in the invention.

### TECHNICAL FIELD

The presently disclosed subject matter generally relates to machine learning methods for analyzing organic semiconductor properties. In particular, the disclosure relates to machine learning methods and systems for modelling intermolecular electronic coupling properties between organic molecules, suitable in applications such as optoelectronic applications. The machine learning methods and systems find utility in a variety of applications, including without intending any limitation optoelectronic applications such as predicting the suitability of organic molecules in organic molecule-based semiconductors.

### BACKGROUND

Molecular crystal structures play a fundamental role in determining the physical, chemical, and mechanical properties of materials.¹⁻⁵ Accurate manipulation and understanding of these structures can lead to the development of materials with tailored properties, opening avenues for innovation and application.⁶⁻¹⁰ Traditional methods for studying and modifying crystal structures often rely on labor-intensive experimental techniques such as X-ray diffraction, nuclear magnetic resonance spectroscopy, and electron microscopy. While these techniques are essential for empirical validation, they can be time-consuming and costly. To overcome these limitations, in silico tools have become integral in materials research, offering the ability to simulate and predict the behavior of materials with high accuracy.¹¹⁻¹⁴ These tools enable researchers to explore a vast space of structural configurations and their corresponding properties, which can significantly accelerate design and discovery processes. Recent advances in machine learning (ML) have further enhanced the capabilities of in-silico tools in materials science.^{11,15-17} ML algorithms can be trained to predict material properties based on large datasets, significantly reducing the time required for simulations and increasing the accuracy of predictions.

The present innovators have previously demonstrated the use of ML models to predict molecular properties from 2D representations of π-conjugated organic molecules and intermolecular properties with 3D representations of molecular dimers.^{18,19} However, for the complete in silico design and discovery of functional molecular crystals, there is a need for ML models that account for the periodicity of crystal structures and intermolecular interactions. While ML potentials provide a way to compute energies of and forces on atoms, most ML potentials are trained on data for single molecules and, therefore, could have limitations with regards to the accurate description of intermolecular interactions that exist in molecular crystals.²⁰⁻²²

To circumvent this challenge, recent works have trained ML potentials on data from crystal structures.²³⁻²⁵ Chen et al. showed that ML potentials derived from graph-based M3GNet models can predict energy, forces and stress in crystals with mean absolute error (MAE) of 0.035 eV/atom, 0.072 eV/Å and 0.41 GPa respectively.²⁴ Notably, the M3GNet model is trained on the data from the Materials Project database,²⁶ which predominantly contains inorganic crystals. ML potentials for the vast chemical space of organic molecular crystals, however, remain limited. Organic materials are generally held together by weak, noncovalent interactions, as opposed to the covalent or ionic (or combinations thereof) bonding found in inorganic materials, presenting distinctive challenges and opportunities.

The design and discovery of functional molecular materials can be greatly accelerated through in silico approaches. Machine learning (ML) models demonstrate significant promise towards the rapid creation, analysis, and manipulation (and re-analysis) of both molecular and crystal structures that are required to hasten materials design and discovery. A critical element of these approaches, however, is that robust ML potentials be developed that allow for accurate predictions of energies and forces acting on atoms within a given crystal. Here, an ML potential trained on 15,000 molecular crystal structures with over 2.5 million crystal geometries is presented.

The present disclosure is directed to an ML potential developed for the rapid prediction of energies, forces, and stress tensors of organic molecular crystals. Notably, the ML potential is trained on 15,000 molecular crystal structures and more than 2.5 million crystal geometries. We demonstrate how the ML potential can be applied to analyses of crystal surface energies and crystal morphologies. To enable the use of the ML potential for in silico discovery of functional molecular crystals, workflows were developed for analyzing crystal surfaces and morphologies, and manipulating and relaxing crystal structures. These workflows may be packaged in various implementing tools, for example OCELOT XtalTransform, an open-access, web-based tool designed to democratize access to the ML model and predictive capabilities.

### SUMMARY

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

In one aspect, the present disclosure is directed to a machine learning-based method for modelling energies, forces, and stress tensors of organic molecular crystals. The method includes inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal. The method further includes, by the machine learning system, converting atomic numbers of the organic molecular crystal into a learnable feature space. The machine learning system further undertakes steps of expanding bond distances of the organic molecular crystal using a basis set comprising second-order derivatives, identifying three-body interactions and corresponding angles for the organic molecular crystal, calculating new bond information of the organic molecular crystal according to bond angles and bond lengths, and iteratively updating one or more of bond, atom, state information of the organic molecular crystal through molecular graph convolution.

In embodiments, the disclosed method further includes a readout phase wherein atom information of the organic molecular crystal is processed by a gated multilayer perceptron to obtain atomic energies of the organic molecular crystal. The machine learning system in embodiments sums the obtained atomic energies to provide a total energy value for the organic molecular crystal and provides force and stress predictions of the organic molecular crystal as derivatives of the total energy value.

In embodiments, the molecular graph representations are three-dimensional noncovalent molecular dimer geometries derived from crystal structures of the at least two organic molecules. The molecular graph representations provide a position in Cartesian coordinates (x, y, z) and atomic number (Z) of all atoms in the noncovalent molecular dimer geometries. The machine learning system may be a graph neural network (GNN).

In another aspect, the present disclosure is directed to a method for training a machine learning (ML) potential for analysis of crystal surface energies and crystal morphologies, including providing a database comprising density functional theory (DFT) crystal relaxation calculations for a plurality of organic molecule crystal structures, wherein unit cells and atomic positions of the plurality of organic molecule crystal structures were relaxed during said DFT crystal relaxation calculations. A training dataset is provided by extracting all organic molecular crystal structures and associated total energies, atomic forces, and stress tensors produced during said relaxation steps of the DFT crystal relaxation calculations and scaling the stress tensors by a factor of 0.1.

In embodiments, the electron-ion interactions of the organic molecular crystals are described by a projector augmented-wave (PAW) method and full periodic boundary conditions. In embodiments, hyperparameters for three-body interactions may include a radial basis expansion value of 6 and an angular expansion value of 7 In embodiments, a 60:20:20 training-validation-test split of the training dataset with average mean square error loss is provided for training the ML potential. One or more training splits, validation splits, and test splits may be provided, each containing 25,000 organic molecular crystal configurations. In embodiments, the ML potential is trained for 20 training epochs, with a batch size of 8. For each training epoch, 10 portions of training data and 1 portion of validation data may be randomly selected.

In another aspect, a machine learning-based method for predicting a surface energy and a crystal morphology of an organic molecular crystal by a trained machine learning (ML) potential as described above is provided, including steps of inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal. Next, a surface slab of the organic molecular crystal having three molecular layers is generated by the machine learning system. A periodic crystal structure of the organic molecular crystal is input into the trained ML potential, and a surface energy of the surface slab is predicted according to the formula:

$$γ = \frac{{E}_{surface} - N {E}_{b u l k}}{2 A}$$

wherein E_{surface} and E_{bulk} are the total energies of the surface and bulk respectively, A is the surface area of the slab, and N is the ratio of atoms in the surface slab to the atoms in bulk.

In embodiments, the method in embodiments further includes, prior to the step of predicting, adding a vacuum of 20 Å normal to the surface slab to avoid interaction with a period image of the organic molecular crystal. The machine learning system then generates a Wulff shape representative of a crystal morphology of the organic molecular crystal according to the predicted surface energy value. The molecular graph representations and machine learning system are as described above.

In yet another aspect, the present disclosure is directed to a machine learning-based method for modelling crystal structure relaxation of an organic molecular crystal, including steps of inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal. The machine learning system, using the trained ML potential as described above, models relaxing the organic molecular crystal structure until a residual Cartesian force component of the organic molecular crystal structure is 0.1 eV Å⁻¹ or less. The ML potential-relaxed organic molecular crystal structure final energy is compared to the DFT dataset crystal relaxation calculation final energy. The molecular graph representations and machine learning system are as described above.

In still yet another aspect, a method is disclosed for modelling manipulation of molecule and cell parameters of an organic molecular crystal. The method includes steps of inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal and further wherein the molecular graph representation provides a position in Cartesian coordinates (x, y, z) and atomic number (Z) of all atoms in the noncovalent molecular dimer geometries. By the machine learning system, Cartesian coordinates of the organic molecular crystal are converted to fractional coordinates. Next, a molecule of the organic molecular crystal structure is extracted, and the machine learning system generates a two-dimensional representation thereof. Next, the machine learning system reconstructs an updated three-dimensional geometry of the organic molecular crystal using a constrained conformer generator function.

In embodiments, the method further includes repeating the steps of converting Cartesian coordinates, extracting a molecule of the organic molecular crystal, generating a two-dimensional representation, and reconstructing a three-dimensional geometry for each molecule of the unit cell. Then, the machine learning system repositions the modified organic molecular crystal structures in their original fractional coordinates within the modified unit cell and displays the modified organic molecular crystal structure.

It will be understood that various details of the presently disclosed subject matter can be changed without departing from the scope of the subject matter disclosed herein. Furthermore, the foregoing description is for the purpose of illustration only, and not for the purpose of limitation.

### BRIEF DESCRIPTION OF THE DRAWINGS

The presently disclosed subject matter will be better understood, and features, aspects and advantages other than those set forth above will become apparent when consideration is given to the following detailed description thereof. Such detailed description makes reference to the following drawings, wherein:

FIG. 1 shows scatter plots showing the correlation between the DFT determined and ML predicted total energy (top), force (center), and stress (bottom) for 520 randomly sampled crystal configurations from the test dataset. In the plot for energy, the total energy of the crystal structure is shown to highlight the range for energy predictions; energy per atom is used during training of the ML potential.

FIG. 2 shows Wulff shape of tetracene obtained using the ML predicted surface energies from Table 1. (Bottom) Experimental crystal morphology of tetracene adapted with permission from Ref.⁴ Copyright 2004 American Chemical Society.

FIG. 3 (Top) Bar plot showing the difference in final energy per atom (ΔE=E_{ML}−E_{DFT}) for structures relaxed with ML workflow and DFT calculations. (Middle) Percent difference in the unit cell final volumes (ΔV=V_{ML}−V_{DFT} and V=V_{DFT}) for structures relaxed with ML workflow and DFT. The labels for the crystal structures correspond to the identifiers in the OCELOT database. (Bottom) Relaxed molecular packings of the TIPS-pentacene (OCELOT ID: com_k01029) crystal derived from the DFT (yellow) and ML (blue) approaches.

FIG. 4 (Top) Workflow for manipulating molecules and the cell parameters of a molecular crystal. (Bottom-left) Experimental crystal structure of TES-Pentacene (OCELOT ID: com_k01066) in slipped-stack placing. (Bottom-right) Brickwork packing crystal structure of TES-Pentacene generated using the crystal manipulation workflow. The experimental crystal structure of TIPS-pentacene (OCELOT ID: com_k01029) was used as the input for the crystal structure manipulation workflow.

FIG. 5 presents a screenshot of the OCELOT XtalTransform web interface.

FIG. 6 shows a screenshot of the 3D Structure tab in OCELOT XtalTransform when a CIF is loaded.

FIG. 7 shows a screenshot of the 2D Editor tab in OCELOT XtalTransform with 2D structure and cell parameters fetched from the 3D Structure tab.

FIG. 8 shows a screenshot of the 2D Editor tab. The molecular structure from FIG. 6 is modified while also changing the unit cell parameters.

FIG. 9 shows a screenshot of the 3D Editor tab shows the 3D crystal structure after modification of molecular structure and relaxation with trained ML model.

FIG. 10 shows a screenshot of the Surface Analysis tab in OCELOT XtalTransform with surface energy predicted by the trained ML model and the computed Wulff surface for the crystal structure in the 3D Structure tab.

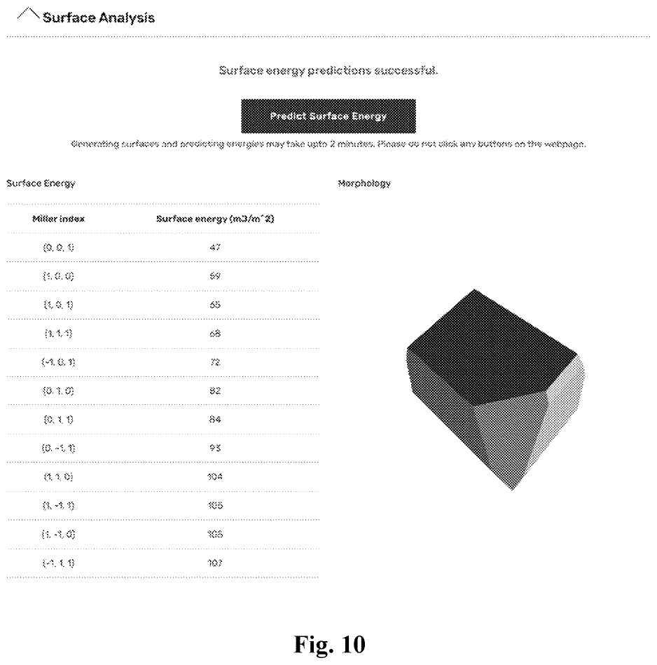

### DETAILED DESCRIPTION

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

At a high level, the present disclosure is directed present disclosure is directed to an ML potential developed for the rapid prediction of energies, forces, and stress tensors of organic molecular crystals. Notably, the ML potential is trained on 15,000 molecular crystal structures and more than 2.5 million crystal geometries, and is adapted to analyses of crystal surface energies and crystal morphologies. To enable the use of the ML potential for in silico discovery of functional molecular crystals, workflows were developed for analyzing crystal surfaces and morphologies, and manipulating and relaxing crystal structures.

### Methods

Dataset. The dataset is derived from density functional theory (DFT) crystal relaxation (geometry optimization) calculations on more than 15,000 experimental crystal structures in the OCELOT (Organic Crystals in Electronic and Light-Oriented Technologies) database.²⁷ The DFT crystal relaxation calculations, were performed using VASP-5.4.4^{28,29} with the Perdew, Burke, and Ernzerhof (PBE)³⁰ exchange-correlation functional. Both the unit cells and atomic positions were relaxed during these calculations. The electron-ion interactions were described by the projector augmented-wave (PAW) method³¹ and full periodic boundary conditions. The kinetic energy cutoff for the plane-wave basis set was set to 600 eV with 50 meV Gaussian smearing applied to the partial occupancies of Kohn-Sham orbitals. To account for van der Waals interaction, Grimme's D3 functional with BJ-damping was used.³² Monkhorst-Pack³³ grids with 64 k-points per Å⁻³ in the reciprocal space were applied to sample the first Brillouin zone. A convergence criterion of 10⁻⁶ eV/atom was used for the self-consistency cycles and the structures were relaxed until the residual Cartesian force components were less than 0.02 eV Å⁻¹ for each atom.

To generate the dataset, all structures produced during the relaxation steps of the DFT calculations were extracted from the output files, along with their associated total energies, atomic forces, and stress tensors. As required for training then ML potential, the stress tensors were scaled by a factor of 0.1. The dataset contains over 2.5 M crystal configurations with energies, forces, and stress.

ML potential. The graph-based M3GNet²⁴ potential was used to predict energies, forces, and stress tensors from crystal configurations. The details on the theory and development of the M3GNet potential can be found in the work from Chen et al.²⁴ Here, we discuss the physical-chemical aspects of the M3GNet potential.

The M3GNet potential architecture begins with a graph representing the positions of all atoms in the crystal. The featurization process converts atomic numbers into a learnable feature space and expands bond distances using a basis set, including second-order derivatives. The potential identifies three-body interactions and corresponding angles, and then calculates new bond information by considering the bonding environment, including angles and bond lengths, and iteratively updates bond, atom, and optional state information through graph convolution. In the readout phase, atom information is processed by a gated multilayer perceptron to obtain atomic energies, which are summed to obtain the total energy. The derivatives of this total energy provide force and stress predictions.

Potential training. The TensorFlow implementation of M3GNet developed by Chen et al. was used as is.²⁴ The same hyperparameters from our previous work were used for the three-body interactions, i.e., the number of radial basis expansions was set to 6, and the number of angular expansions was set to 7.¹⁹ This hyperparameter set was observed to perform better than the default hyperparameters. A 60:20:20 training-validation-test split of the dataset was used with average mean square error (MSE) loss for training the ML potential. With the dataset being over 10 GB in size, chunks of the training (50), validation (16), and test splits (16) were made, each with 25,000 crystal configurations. The ML potentials were trained for 20 epochs, with a batch size of 8, and used the Adam optimizer³⁴ with a learning rate of 0.001. To circumvent the large memory requirements for training, 10 random chunks of training and a random chunk of validation data were selected during each epoch. ML potential training was performed in TensorFlow version 2.8.1 and used Cuda 11.4 for GPU acceleration on a single NVIDIA Tesla V100 GPU.³⁵ Each training epoch took 5 hours

### Results and Discussion

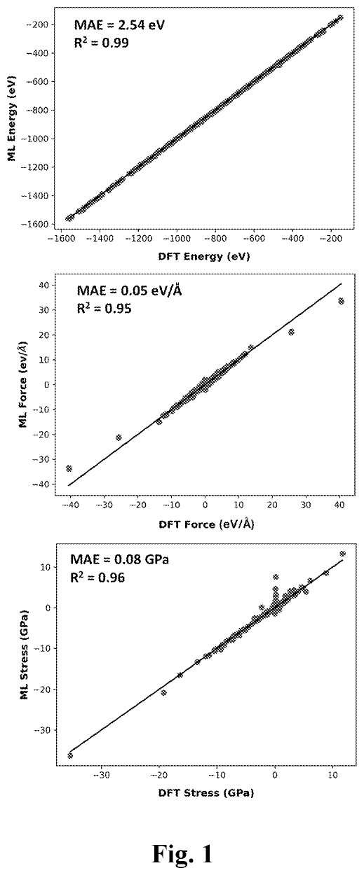

The ML potential, based on M3GNet, was trained on the OCLEOT crystal relaxation vl dataset, which consists of more than 2.5 million crystal configurations with DFT computed energies, forces on each atom, and stress tensors from over 15k crystal structures in the OCELOT database.²⁷ The input to the ML potential is the crystal configuration, which is then transformed into a graph for predictions (see Methods for a detailed description). The trained potential has a mean absolute error (MAE) of 0.022 eV/atom, 0.056 eV/Å, and 0.076 GPa for energy, force, and stress, respectively. The trained ML potential can predict these quantities over a large range of the respective values, as shown in FIG. 1. The performance of this potential is better than the reported potential trained on inorganic systems (0.035 eV/atom, 0.072 eV/Å and 0.41 GPa for energy, forces and stress respectively).24 However, it is important to note that these values correspond to different datasets. The trained ML potential performance is evaluated on the OCELOT dataset, while the previously reported potential was trained and tested on an inorganic dataset. The inorganic materials-trained model has not been tested on our dataset, so a direct comparison of absolute error values should be interpreted within this context. However, the improved performance of the trained ML potential could be attributed to the number of atom types used during the training of the ML potential. In contrast to the M3GNet potential from Chen et al., which can handle 89 atom types, our trained ML potential is trained on only 14 atom types, which includes C, N, O, F, S, Cl, Br, Se, P, Si, B, As, Te, I, and H. Previously reported ML potentials^{22,25} for organic systems are only trained on C, H, N, O, F, Cl, and S atom types, which make the trained ML potential presented here applicable over a larger chemical space

Surface energy and morphology predictions. The determination of surface energy and crystal morphology is critical in understanding the properties and functions of molecular crystals. As the trained ML potential can predict energies, we designed a workflow to predict surface energies and crystal morphologies. The initial step of the workflow involves the generation of surface slabs. The Python package Ogre is used to generate the surface slabs with three molecular layers.³⁶ Marom and coworkers showed that three layers are sufficient to produce reliable Wulff surfaces.³⁶ A vacuum of 20 Å is added normal to the slab surface to avoid spurious interaction with the period image. The total energy of the bulk crystal and the surface slabs is predicted with the trained ML potential. The input to the potential is the periodic crystal structure. The surface energy (y) is evaluated using

$$γ = \frac{{E}_{surface} - N {E}_{b u l k}}{2 A}$$

where E_{surface} and E_{bulk} are the total energies of the surface and bulk respectively, A is the surface area of the slab, and N is the ratio of atoms in the surface slab to the atoms in bulk. Further, we use surface energy to generate the Wulff shape, which represents crystal morphology. The Wulff shapes are generated with the WulffShape module in the Python package pymatgen.²⁴

TABLE 1
Surface energies for tetracene (OCELOT ID: csd_TETCEN) in
mJ/m². The The DFT-derived values are obtained using the PBE +
TS and PBE + MBD functionals (from Ref.³⁶) Surface structures
were generated using the Ogre package.³⁶ The ML predictions
are based on slabs containing three molecular layers. The ML
model is trained on the data generated using PBD + D3 functional.

| Miller | PBE + TS | PBE + MBD | ML |
|---|---|---|---|
| Index | (mJ/m²) | (mJ/m²) | (mJ/m²) |
| 001 | 114.76 | 89.2 | 38.02 |
| 100 | 156.54 | 112.78 | 51.13 |
| 1-10 | 164.63 | 117.29 | 52.24 |
| 110 | 163.19 | 118.81 | 50.74 |
| 101 | 160.89 | 120.91 | 54.99 |
| −111 | 172.19 | 124.99 | 57.66 |
| 111 | 167.54 | 125.89 | 54.67 |
| 1-11 | 178.20 | 131.35 | 60.30 |
| −1-11 | 178.16 | 131.65 | 59.49 |
| −101 | 179.18 | 132.28 | 64.02 |
| 010 | 200.23 | 147.83 | 67.84 |
| 011 | 199.16 | 150.16 | 68.96 |
| 0-11 | 218.47 | 165.07 | 77.21 |

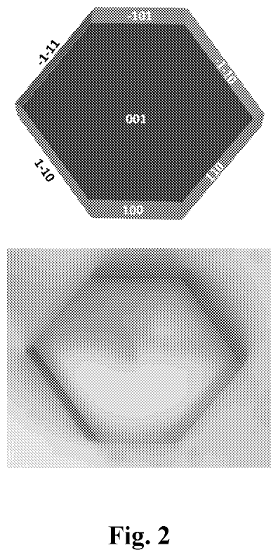

As an example, we used the ML potential derived here to model and predict the surface energies of tetracene. Tetracene was chosen as there exists reported surface energies computed for the system with DFT.³⁶ The surfaces for tetracene crystal (OCELOT ID: csd_TETCEN) are generated using the Python package Orge. While absolute values of surface energy may vary depending on parameters such as the DFT functional, plane-wave energy cutoff, and van der Waals corrections, the trends in the surface energy remain consistent.³⁶ Although the ML potential generally predicts lower surface energy values compared to DFT, the relative trends among different surfaces are largely in agreement (see Table 1). The Wulff shape generated from surface energy estimates of the trained ML potential is in line with experimental observations, as shown in FIG. 2.

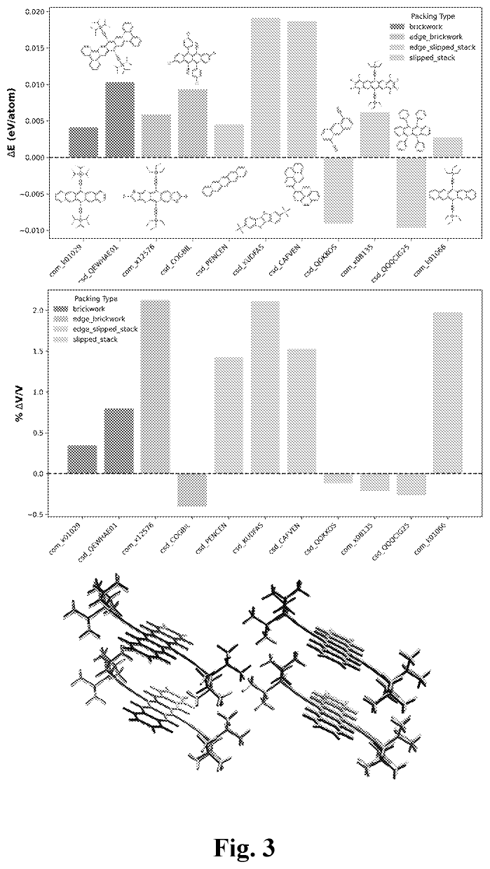

Crystal structure relaxation. A computational bottleneck for many in silico molecular crystal structure discovery and design is the crystal relaxation (geometry optimization) process, which requires accurate estimates of forces acting on atoms. As the trained ML potential predicts forces and energies, we designed a workflow to relax both the unit cell parameters and atomic coordinates of a molecular crystal structure. In the workflow, the crystal structures are relaxed using the trained ML potential and the Relaxer class from the M3GNet package.²⁴ The BFGS optimizer³⁷ is used with a maximum of 2500 steps. The structure is relaxed until the residual Cartesian force components are less than 0.1 eV Å⁻¹. To test the effectiveness of the structure relaxation workflow, we relaxed experimental crystal structures with the trained ML potential, and the final cell parameters and energies were compared to the DFT relaxation data from the OCELOT database. Known organic semiconducting materials with varying crystal packing were selected for the comparison. The differences in final energies of the relaxed structures from the ML workflow and DFT are shown in FIG. 3 and Table 2. All ML-predicted energies fall within a MAE of 0.022 eV/atom when compared to the DFT results. While most of the ML predictions are underestimated, as observed in the surface energy predictions, there is a greater variation observed for slip-stack packing configurations. The percent volume change (see FIG. 3) for the final structures shows similar trends with brickwork packing configurations, providing structures of similar cell volumes as DFT calculations. However, the final atomic positions of all the ML relaxed structures do not deviate significantly from the DFT structure as shown in FIG. 3. The deviations from DFT energy and volume observed for the ML relaxed structures could be attributed to the force predictions from the trained ML potential, which have an MAE of 0.056 eV/Å, the same order of magnitude as the convergence criterion of 0.02 eV Å⁻¹ for DFT relaxation calculations. Thus, using ML potential forces for structure relaxation may not yield the exact DFT relaxed crystal structure in terms of energy and volume, though it can provide several orders of magnitude speed up when compared to DFT approaches and produce reasonable crystal geometries for further analyses. For instance, the DFT crystal structure relaxation calculation for the crystal structure of TIPS pentacene (OCLEOT ID: com_k01029) takes approximately 5 hours on 16 cores of Intel 6252 Cascade with 48 GB RAM, while the ML structure relaxation requires less than one minute on a single core Intel Xenon E3-1241 with 4 GB RAM.

TABLE 2
Cell parameters, volume, and energy for TIPS pentacene (OCELOT
ID: com_k01029). The DFT and ML results are for the relaxed
crystal structures derived through the respective approaches.

| | a | b | c | a | b | g | Volume | Energy |
|---|---|---|---|---|---|---|---|---|
| Structure | (Å) | (Å) | (Å) | (degree) | (degree) | (degree) | (Å³) | (eV/atom) |
| Crystal | 7.57 | 7.75 | 16.84 | 89 | 78 | 84 | 961 | — |
| DFT | 7.39 | 7.66 | 16.59 | 90 | 80 | 85 | 919 | −6.121 |
| ML | 7.43 | 7.60 | 16.67 | 90 | 79 | 85 | 922 | −6.117 |

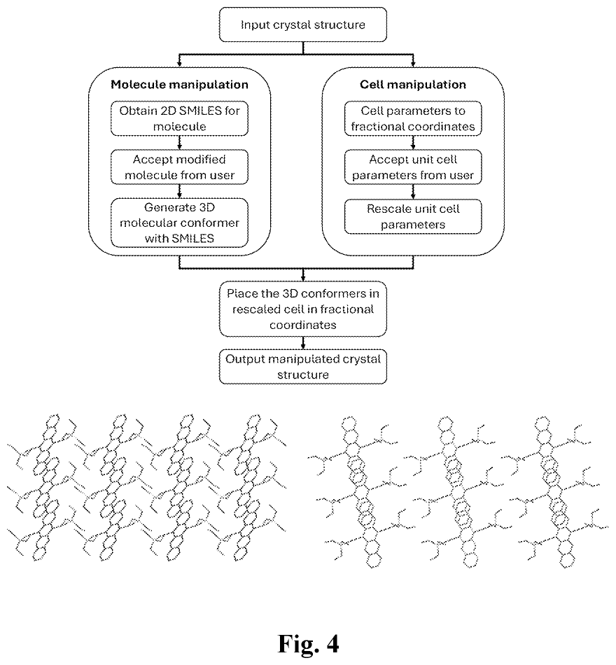

Crystal structure manipulation. Tools to edit molecular crystal structures are necessary for in silico design as they allow one to modify a known system and then predict the properties of the updated structure. We developed a workflow for interactively editing both the molecular structures and unit cell parameters. As shown in FIG. 4, the workflow performs crystal structure modifications in three independent steps. These steps allow for flexible editing of both the unit cell parameters and molecular structures while preserving the overall crystal arrangement. The Python packages OCELOT API²⁷ and RDKit³⁸ and JSmol³⁹ are used for interactive 2D molecular structure editing while simultaneously displaying and updating the 3D crystal structure

First, the Cartesian coordinates of the crystal are converted to fractional coordinates, ensuring that any transformation of the unit cell does not affect the relative positions of the molecules. Based on user input, the unit cell parameters are modified accordingly. Independently, the molecules from the crystal structure are extracted and their 2D representations are generated using the OCELOT API. Users can modify the molecular structure interactively in JSmol, where the SMILES representation of the edited molecule is captured. The updated 3D geometry is then reconstructed using the constrained conformer generator function in RDKit, which is the most computationally demanding step of the workflow. This process is repeated for all molecules within the unit cell. Finally, the modified molecules are placed back at their original fractional coordinates within the transformed unit cell, and the updated crystal structure is displayed in JSmol for visualization. As a demonstration of our workflow, we generated an in silico brickwork packing crystal structure of TES-pentacene. This is notable because TES-pentacene is experimentally observed to form a slip-stack packing arrangement (OCELOT ID: com_k01066). To achieve this transformation, we utilized the brickwork packing crystal structure of TIPS-pentacene (OCELOT ID: com_k01029) as input. Our workflow involved removing the methyl groups from the sidechains of the 2D TIPS-pentacene structure, effectively generating the TES-pentacene structure as shown in FIG. 4.

### Examples

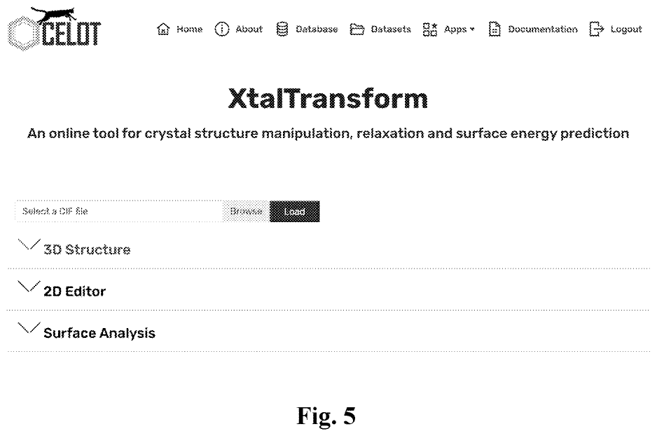

In one specific embodiment, the disclosed workflows were implemented on the OCELOT XtalTransform web interface (Oscar.as.uky.edu/xtaltransform). See FIG. 5.

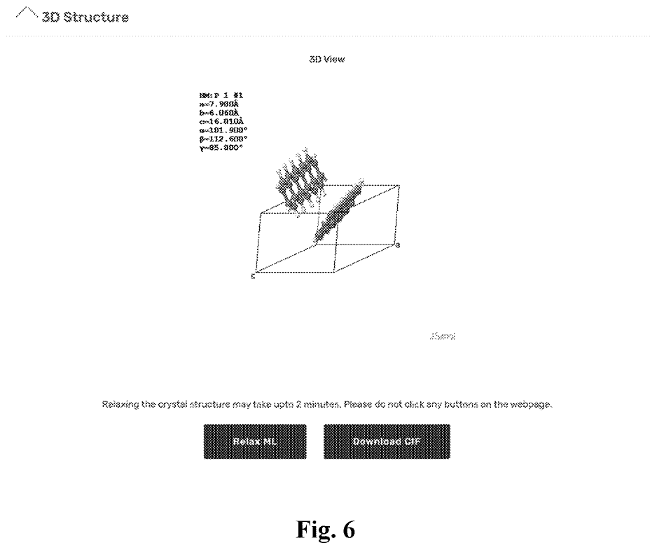

Loading and Relaxing Crystal Structure. The first step involves uploading the crystallographic information file (CIF) of the crystal structure. Click the “Browse” button for a dialog box to appear. Users can select a CIF from their PC to edit, relax, or compute the surface energy. Once the CIF is selected, click “Load” to upload the structure to OCELOT XtalTransform. If users want to try the tool and do not have a CIF, clicking “Load” will automatically load a sample CIF. After the crystal structure is loaded, the 3D structure tab is populated with the 3D visualization for the structure as shown in FIG. 6.

The 3D Structure tab provides the option to Relax the structure with the trained ML model. To use the functionality, click “Relax ML”. The time required to relax depends on the number of atoms in the unit cell. The larger the number of atoms, the longer the time to relax the structure. After the relaxation, the relaxed structure will replace the initial structure in the 3D Structure tab. Any crystal structure displayed in the 3D Structure can be downloaded by clicking “Download CIF”. Note that there is no need to relax the crystal structure before modifying it. It is good practice to relax the crystal structure after any modification.

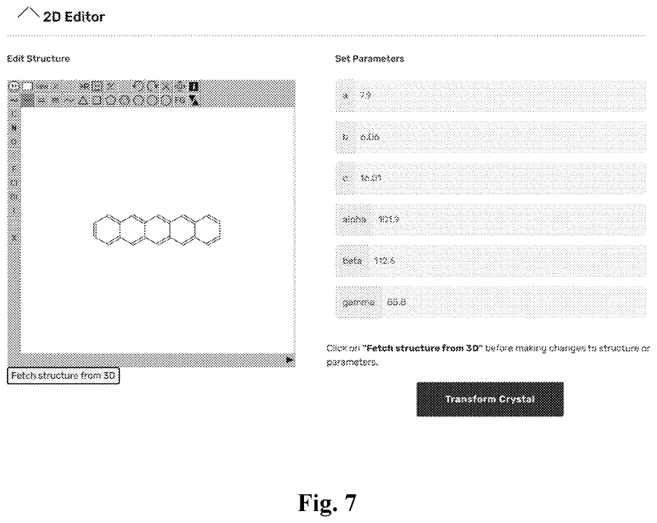

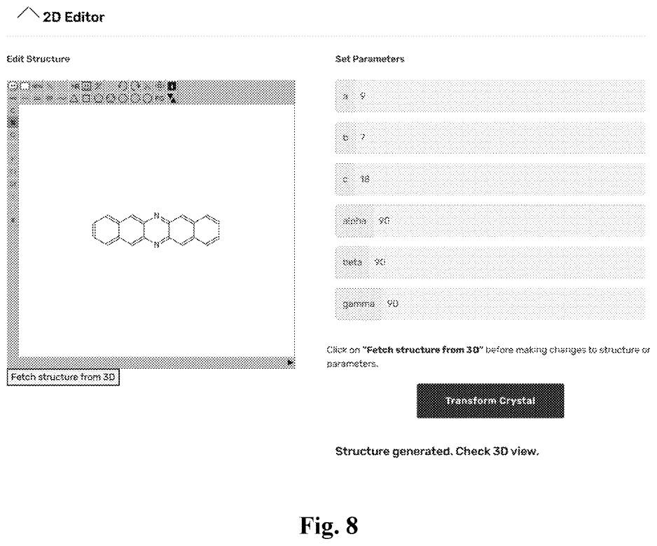

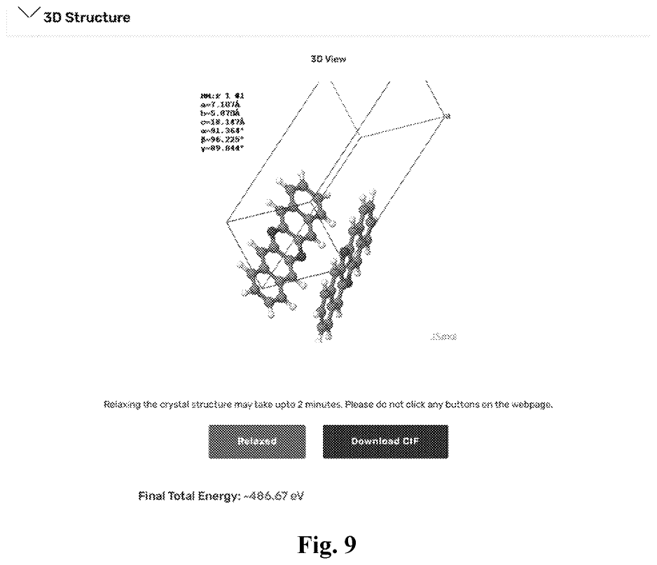

Modifying Crystal Structure. After a CIF has been loaded, the crystal structure can be directly edited under the 2D Editor tab. To start the modification process, click “Fetch structure from 3D” under the structure editor (FIG. 6). This will fetch the 2D molecular structure of the crystal structure displayed under the 3D Structure tab. Users can transform the 2D molecular structure using the editor (FIG. 8). The cell parameters are directly loaded from the crystal structure displayed under the 3D Structure tab, as shown in FIG. 7. To change the parameters, users can type in the required values for the cell parameters. To generate the transformed crystal structure, click “Transform Crystal”. The crystal structure generation will take a few minutes. The processing time depends on the number of new rotatable bonds in transformed 2D. The addition of large alkyl chains will increase the time to generate the transformed crystal (FIG. 9).

Surface Energy and Morphology. After loading a CIF, the surface energy and morphology calculation can be initiated under the Surface Analysis tab. This calculation can be carried out without crystal structure relaxation and transformation. However, it is recommended that relaxation be performed on the transformed crystal structure before the surface analysis. The Surface Analysis tab displays the surface energy for low-index surfaces and the Wulff shape generated from the surface energy calculations. The surface analysis can be initiated by clicking “Predict Surface Energy”. The processing time depends on the number of atoms and the symmetry of the crystal. A sample surface analysis calculation is shown in FIG. 8.

### Conclusions.

To aid in the in silico design and discovery of organic molecular crystals, we present the development of an ML potential trained on more than 15,000 crystal structures, containing 14 atom types (C, N, O, F, S, Cl, Br, Se, P, Si, B, As, Te, I, and H), and 2.5 million crystal geometries. The ML potential can predict the energies and forces of atoms in a molecular crystal structure with an MAE of 0.022 eV/atom for energy and 0.056 eV/Å for force. With the ML potential, we then created workflows to predict crystal surface energies and morphologies and to relax molecular crystal structures. Further, we implemented a workflow for the manipulation of molecular crystal structures. By this work, it is possible to upload a molecular crystal structure to visualize and transform the crystal with the crystal manipulation workflow. The interface allows the transformed crystal to undergo structure relaxation using the trained ML potential. Moreover, the surface energy and morphology workflow can be performed interactively.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Any and all patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Unless otherwise indicated, all numbers expressing quantities or values are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about” is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

### REFERENCES

ADDIN ZOTERO BIBL {“uncited”: [ ], “omitted”: [ ], “custom”: [ ]} CSL_BIBLIOGRAPHY (1) Datta, S.; Grant, D. J. W. Crystal Structures of Drugs: Advances in Determination, Prediction and Engineering. Nat. Rev. Drug Discov. 2004, 3 (1), 42-57. https://doi.org/10.1038/nrd1280.
(2) Song, Y.; Pan, G.; Zhang, C.; Wang, C.; Xu, B.; Tian, W. Organic Luminescent Crystals: Role of Packing Structures and Optical Properties. Mater. Chem. Front. 2023, 7 (21), 5104-5119. https://doi.org/10.1039/D3QM00494E.
(3) Bernstein, J. Crystal Growth, Polymorphism and Structure-Property Relationships in Organic Crystals. J. Phys. Appl. Phys. 1993, 26 (8B), B66. https://doi.org/10.1088/0022-3727/26/8B/010.
(4) Molecular Crystals and Molecules; 2012.
(5) Hemley, R. J.; Dera, P. Molecular Crystals. Rev. Mineral. Geochem. 2000, 41 (1), 335-419. https://doi.org/10.2138/rmg.2000.41.12.
(6) Ramamurthy, V.; Venkatesan, K. Photochemical reactions of organic crystals. ACS Publications. https://doi.org/10.1021/cr00078a009.
(7) Corpinot, M. K.; Bučar, D.-K. A Practical Guide to the Design of Molecular Crystals. Cryst. Growth Des. 2019, 19 (2), 1426-1453. https://doi.org/10.1021/acs.cgd.8b00972.
(8) Thompson, A. J.; Orue, A. I. C.; Nair, A. J.; Price, J. R.; McMurtrie, J.; Clegg, J. K. Elastically Flexible Molecular Crystals. Chem. Soc. Rev. 2021, 50 (21), 11725-11740. https://doi.org/10.1039/DICS00469G.
(9) Naumov, P.; Chizhik, S.; Panda, M. K.; Nath, N. K.; Boldyreva, E. Mechanically Responsive Molecular Crystals. Chem. Rev. 2015, 115 (22), 12440-12490. https://doi.org/10.1021/acs.chemrev.5b00398.
(10) Wang, C.; Dong, H.; Jiang, L.; Hu, W. Organic Semiconductor Crystals. Chem. Soc. Rev. 2018, 47 (2), 422-500. https://doi.org/10.1039/C7CS00490G.
(11) Bhat, V.; Callaway, C. P.; Risko, C. Computational Approaches for Organic Semiconductors: From Chemical and Physical Understanding to Predicting New Materials. Chem. Rev. 2023, 123 (12), 7498-7547. https://doi.org/10.1021/acs.chemrev.2c00704.
(12) Jain, A.; Shin, Y.; Persson, K. A. Computational Predictions of Energy Materials Using Density Functional Theory. Nat. Rev. Mater. 2016, 1 (1), 1-13. https://doi.org/10.1038/natrevmats.2015.4.
(13) Gómez-Bombarelli, R.; Aguilera-Iparraguirre, J.; Hirzel, T. D.; Duvenaud, D.; Maclaurin, D.; Blood-Forsythe, M. A.; Chae, H. S.; Einzinger, M.; Ha, D.-G.; Wu, T.; Markopoulos, G.; Jeon, S.; Kang, H.; Miyazaki, H.; Numata, M.; Kim, S.; Huang, W.; Hong, S. I.; Baldo, M.; Adams, R. P.; Aspuru-Guzik, A. Design of Efficient Molecular Organic Light-Emitting Diodes by a High-Throughput Virtual Screening and Experimental Approach. Nat. Mater. 2016, 15 (10), 1120-1127. https://doi.org/10.1038/nmat4717.
(14) Sadybekov, A. V.; Katritch, V. Computational Approaches Streamlining Drug Discovery. Nature 2023, 616 (7958), 673-685. https://doi.org/10.1038/s41586-023-05905-z.
(15) Green, J. D.; Fuemmeler, E. G.; Hele, T. J. H. Inverse Molecular Design from First Principles: Tailoring Organic Chromophore Spectra for Optoelectronic Applications. J. Chem. Phys. 2022, 156 (18), 180901. https://doi.org/10.1063/5.0082311.
(16) Sun, W.; Zheng, Y.; Yang, K.; Zhang, Q.; Shah, A. A.; Wu, Z.; Sun, Y.; Feng, L.; Chen, D.; Xiao, Z.; Lu, S.; Li, Y.; Sun, K. Machine Learning-Assisted Molecular Design and Efficiency Prediction for High-Performance Organic Photovoltaic Materials. Sci. Adv. 2019, 5 (11), eaay4275. https://doi.org/10.1126/sciadv.aay4275.
(17) Mahmood, A.; Wang, J.-L. Machine Learning for High Performance Organic Solar Cells: Current Scenario and Future Prospects. Energy Environ. Sci. 2021, 14 (1), 90-105. https://doi.org/10.1039/DOEE02838J.
(18) Bhat, V.; Sornberger, P.; Pokuri, B. S. S.; Duke, R.; Ganapathysubramanian, B.; Risko, C. Electronic, Redox, and Optical Property Prediction of Organic π-Conjugated Molecules through a Hierarchy of Machine Learning Approaches. Chem. Sci. 2023, 14 (1), 203-213. https://doi.org/10.1039/D2SC04676H.
(19) Bhat, V.; Ganapathysubramanian, B.; Risko, C. Rapid Estimation of the Intermolecular Electronic Couplings and Charge-Carrier Mobilities of Crystalline Molecular Organic Semiconductors through a Machine Learning Pipeline. Mar. 8, 2023. https://doi.org/10.26434/chemrxiv-2023-rvzmv.
(20) Unke, O. T.; Meuwly, M. PhysNet: A Neural Network for Predicting Energies, Forces, Dipole Moments, and Partial Charges. J. Chem. Theory Comput. 2019, 15 (6), 3678-3693. https://doi.org/10.1021/acs.jctc.9b00181.
(21) Schütt, K. T.; Sauceda, H. E.; Kindermans, P.-J.; Tkatchenko, A.; Müller, K.-R. SchNet-A Deep Learning Architecture for Molecules and Materials. J. Chem. Phys. 2018, 148 (24), 241722. https://doi.org/10.1063/1.5019779.
(22) Smith, J. S.; Isayev, O.; Roitberg, A. E. ANI-1: An Extensible Neural Network Potential with DFT Accuracy at Force Field Computational Cost. Chem. Sci. 2017, 8 (4), 3192-3203. https://doi.org/10.1039/C6SC05720A.
(23) Anstine, D. M.; Isayev, O. Machine Learning Interatomic Potentials and Long-Range Physics. J. Phys. Chem. A 2023, 127 (11), 2417-2431. https://doi.org/10.1021/acs.jpca.2c06778.
(24) Chen, C.; Ong, S. P. A Universal Graph Deep Learning Interatomic Potential for the Periodic Table. Nat. Comput. Sci. 2022, 2 (11), 718-728. https://doi.org/10.1038/s43588-022-00349-3.
(25) Žugec, I.; Geilhufe, R. M.; Lončarić, I. Global Machine Learning Potentials for Molecular Crystals. J. Chem. Phys. 2024, 160 (15), 154106. https://doi.org/10.1063/5.0196232.
(26) Jain, A.; Ong, S. P.; Hautier, G.; Chen, W.; Richards, W. D.; Dacek, S.; Cholia, S.; Gunter, D.; Skinner, D.; Ceder, G.; Persson, K. A. Commentary: The Materials Project: A Materials Genome Approach to Accelerating Materials Innovation. APL Mater. 2013, 1 (1), 011002. https://doi.org/10.1063/1.4812323.
(27) Ai, Q.; Bhat, V.; Ryno, S. M.; Jarolimek, K.; Sornberger, P.; Smith, A.; Haley, M. M.; Anthony, J. E.; Risko, C. OCELOT: An Infrastructure for Data-Driven Research to Discover and Design Crystalline Organic Semiconductors. J. Chem. Phys. 2021, 154 (17), 174705. https://doi.org/10.1063/5.0048714.
(28) Kresse, G.; Furthmüller, J. Efficiency of Ab-Initio Total Energy Calculations for Metals and Semiconductors Using a Plane-Wave Basis Set. Comput. Mater. Sci. 1996, 6 (1), 15-50. https://doi.org/10.1016/0927-0256 (96) 00008-0.
(29) Kresse, G.; Hafner, J. Ab Initio Molecular Dynamics for Liquid Metals. Phys. Rev. B 1993, 47 (1), 558-561. https://doi.org/10.1103/PhysRevB.47.558.
(30) Perdew, J. P.; Burke, K.; Ernzerhof, M. Generalized Gradient Approximation Made Simple. Phys. Rev. Lett. 1996, 77 (18), 3865-3868. https://doi.org/10.1103/PhysRevLett.77.3865.
(31) Blöchl, P. E. Projector Augmented-Wave Method. Phys. Rev. B 1994, 50 (24), 17953-17979. https://doi.org/10.1103/PhysRevB.50.17953.
(32) Grimme, S.; Ehrlich, S.; Goerigk, L. Effect of the Damping Function in Dispersion Corrected Density Functional Theory. J. Comput. Chem. 2011, 32 (7), 1456-1465. https://doi.org/10.1002/jcc.21759.
(33) Monkhorst, H. J.; Pack, J. D. Special Points for Brillouin-Zone Integrations. Phys. Rev. B 1976, 13 (12), 5188-5192. https://doi.org/10.1103/PhysRevB.13.5188.
(34) Kingma, D. P.; Ba, J. Adam: A Method for Stochastic Optimization. arXiv Jan. 29, 2017. http://arxiv.org/abs/1412.6980 (accessed 2024 Jun. 21).
(35) Nickolls, J.; Buck, I.; Garland, M.; Skadron, K. Scalable Parallel Programming with CUDA: Is CUDA the Parallel Programming Model That Application Developers Have Been Waiting For? Queue 2008, 6 (2), 40-53. https://doi.org/10.1145/1365490.1365500.
(36) Yang, S.; Bier, I.; Wen, W.; Zhan, J.; Moayedpour, S.; Marom, N. Ogre: A Python Package for Molecular Crystal Surface Generation with Applications to Surface Energy and Crystal Habit Prediction. J. Chem. Phys. 2020, 152 (24), 244122. https://doi.org/10.1063/5.0010615.
(37) Fletcher, R. Practical Methods of Optimization, 2nd ed.; Wiley: Chichester; New York, 1987.
(38) Greg Landrum; Paolo Tosco; Brian Kelley; Ricardo Rodriguez; David Cosgrove; Riccardo Vianello; sriniker; gedeck; Gareth Jones; NadineSchneider; Eisuke Kawashima; Dan Nealschneider; Andrew Dalke; Matt Swain; Brian Cole; Samo Turk; Aleksandr Savelev; Alain Vaucher; Maciej Wójcikowski; Ichiru Take; Vincent F. Scalfani; Rachel Walker; Kazuya Ujihara; Daniel Probst; guillaume godin; Axel Pahl; tadhurst-cdd; Juuso Lehtivarjo; Francois Berenger; jasondbiggs. Rdkit/Rdkit: 2024_03_3 (Q1 2024) Release, 2024. https://doi.org/10.5281/ZENODO.591637.
(39) Hanson, R. M.; Prilusky, J.; Renjian, Z.; Nakane, T.; Sussman, J. L. JSmol and the Next-Generation Web-Based Representation of 3D Molecular Structure as Applied to Proteopedia. Isr. J. Chem. 2013, 53 (3-4), 207-216. https://doi.org/10.1002/ijch.201300024.

What is claimed:

1. A machine learning-based method for modelling energies, forces, and stress tensors of organic molecular crystals, comprising:
inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal;
by the machine learning system, converting atomic numbers of the organic molecular crystal into a learnable feature space;
by the machine learning system, expanding bond distances of the organic molecular crystal using a basis set comprising second-order derivatives;
by the machine learning system, identifying three-body interactions and corresponding angles for the organic molecular crystal;
by the machine learning system, calculating new bond information of the organic molecular crystal according to bond angles and bond lengths; and
by the machine learning system, iteratively updating one or more of bond, atom, state information of the organic molecular crystal through molecular graph convolution.

2. The method of claim 1, further comprising a readout phase wherein atom information of the organic molecular crystal is processed by a gated multilayer perceptron to obtain atomic energies of the organic molecular crystal.

3. The method of claim 2, further comprising, by the machine learning system, summing the obtained atomic energies to provide a total energy value for the organic molecular crystal.

4. The method of claim 3, further comprising, by the machine learning system, providing force and stress predictions of the organic molecular crystal as derivatives of the total energy value.

5. The method of claim 1, wherein the molecular graph representations are three-dimensional noncovalent molecular dimer geometries derived from a crystal structure of the organic molecular crystal, providing a position in Cartesian coordinates (x, y, z) and atomic number (Z) of all atoms in the noncovalent molecular dimer geometries.

6. The method of claim 1, wherein the machine learning system is a graph neural network (GNN).

7. A method for training a machine learning (ML) potential for analysis of crystal surface energies and crystal morphologies, comprising:
providing a database comprising density functional theory (DFT) crystal relaxation calculations for a plurality of organic molecule crystal structures, wherein unit cells and atomic positions of the plurality of organic molecule crystal structures were relaxed during said DFT crystal relaxation calculations;
providing a training dataset by extracting all organic molecular crystal structures and associated total energies, atomic forces, and stress tensors produced during said relaxation steps of the DFT crystal relaxation calculations; and
scaling stress tensors of said organic molecular crystal structures by a factor of 0.1.

8. The method of claim 7, wherein electron-ion interactions of the organic molecular crystals are described by a projector augmented-wave (PAW) method and full periodic boundary conditions.

9. The method of claim 7, wherein hyperparameters for three-body interactions comprise:
a radial basis expansion value of 6; and
an angular expansion value of 7.

10. The method of claim 9, further comprising providing a 60:20:20 training-validation-test split of the training dataset with average mean square error loss for training the ML potential.

11. The method of claim 7, further comprising providing one or more training splits, validation splits, and test splits, each containing 25,000 organic molecular crystal configurations.

12. The method of claim 11, further comprising training the ML potential for 20 training epochs, with a batch size of 8.

13. The method of claim 12, further comprising, for each training epoch, randomly selecting 10 portions of training data and 1 portion of validation data.

14. A machine learning-based method for predicting a surface energy and a crystal morphology of an organic molecular crystal by a trained machine learning (ML) potential according to the method of claim 7, comprising:
inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal;
by the machine learning system, generating a surface slab of the organic molecular crystal having three molecular layers;
inputting a periodic crystal structure of the organic molecular crystal to the trained ML potential;
predicting a surface energy of the surface slab according to the formula:
$$γ = \frac{{E}_{surface} - N {E}_{b u l k}}{2 A}$$
wherein:
E_{surface} and E_{bulk} are the total energies of the surface and bulk respectively, A is the surface area of the slab, and N is the ratio of atoms in the surface slab to the atoms in bulk.

15. The method of claim 14, further comprising prior to the step of predicting:
by the machine learning system, adding a vacuum of 20 Å normal to the surface slab to avoid interaction with a period image of the organic molecular crystal.

16. The method of claim 15, further comprising, by the machine learning system, generating a Wulff shape representative of a crystal morphology of the organic molecular crystal according to the predicted surface energy value.

17. The method of claim 14, wherein the molecular graph representations are three-dimensional noncovalent molecular dimer geometries derived from a crystal structure of the organic molecular crystal and providing a position in Cartesian coordinates (x, y, z) and atomic number (Z) of all atoms in the noncovalent molecular dimer geometries.

19. A machine learning-based method for modelling crystal structure relaxation of an organic molecular crystal, comprising:
inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal;
by the machine learning system using the trained ML potential of claim 7, relaxing the organic molecular crystal structure until a residual Cartesian force component of the organic molecular crystal structure is 0.1 eV Å⁻¹ or less; and
comparing the ML potential-relaxed organic molecular crystal structure final energy to the DFT dataset crystal relaxation calculation final energy.

20. The method of claim 19, wherein the molecular graph representations are three-dimensional noncovalent molecular dimer geometries derived from a crystal structure of the organic molecular crystal providing a position in Cartesian coordinates (x, y, z) and atomic number (Z) of all atoms in the noncovalent molecular dimer geometries.

21. A machine learning-based method for modelling manipulation of molecule and cell parameters of an organic molecular crystal, comprising:
inputting into a machine learning system a synthetically generated graph representation of an organic molecular crystal, wherein the graph representation is a molecular graph representation in which each node corresponds to an atom of the organic molecular crystal and each edge corresponds to a chemical bond between atoms of the organic molecular crystal and further wherein the molecular graph representation provides a position in Cartesian coordinates (x, y, z) and atomic number (Z) of all atoms in the noncovalent molecular dimer geometries;
by the machine learning system, converting the Cartesian coordinates of the organic molecular crystal to fractional coordinates;
by the machine learning system, extracting a molecule of the organic molecular crystal and generating a two-dimensional representation thereof; and
reconstructing an updated three-dimensional geometry of the organic molecular crystal using a constrained conformer generator function.

22. The method of claim 21, further comprising repeating the steps of converting Cartesian coordinates, extracting a molecule of the organic molecular crystal, generating a two-dimensional representation, and reconstructing a three-dimensional geometry for each molecule of the unit cell.

23. The method of claim 22, further comprising by the machine learning system re-positioning the modified organic molecular crystal structures in their original fractional coordinates within the modified unit cell; and
displaying the modified organic molecular crystal structure.
